(19) United States
(12) Patent Application Publication
(10) Pub. No.: US 2018/0362456 A1
(43) Pub. Date: Dec. 20, 2018

(54) NEW COMPOUNDS WITH ANTIOXIDANT AND ANTIAGING ACTIVITY

(71) Applicant: EMS S/A, Hortolândia (BR)

(72) Inventors: Giancarlo SANTUS, Milão (IT); Silvana MASIERO, Hortolândia (BR); Leticia Khater COVESI, Hortolândia (BR)

(21) Appl. No.: 16/060,359

(22) PCT Filed: Dec. 6, 2016
(86) PCT No.: PCT/BR2016/050315

§ 371 Date: Jun. 7, 2018

Related U.S. Application Data
(60) Provisional application No. 62/264,159, filed on Dec. 7, 2015.

(51) Int. Cl. C07C 323/58; C07D 207/28; C07C 323/59; C07D 475/04; A61K 8/46; A61K 8/49; A61Q 19/08; A61P 39/06

(52) U.S. Cl. CPC C07C 323/58; C07D 207/28; C07C 323/59; C07D 475/04; C07C 2603/52; A61K 8/4913; A61K 8/494; A61Q 19/08; A61P 39/06; A61K 8/46

(57) ABSTRACT

The present invention describes new derivatives of S-allylcysteine with antioxidant and antiaging activity. Said derivatives can be used alone or in a combined formulation with other compounds with known activity.

### FIELD OF THE INVENTION

The present invention relates to compounds with antioxidant, antiaging and antiinflammatory activity, particularly S-allylcysteine derivatives. Said compounds may be used alone or combined with other active compounds and eventual cosmetically acceptable excipients to treat and/or prevent the effects of skin aging, providing antiaging benefits.

### BACKGROUND

Skin aging is a multisystem degenerative process that involves skin and skin support. Skin aging is mainly the result of genetic predisposition (physiological aging, or intrinsic aging) and the physiological reaction to environmental stresses, such as exposure to UV irradiation (sun), pollution, smoking etc. (extrinsic aging).

The biological mechanism of cutaneous aging is characterized by an alteration of the dermis with appearance of skin fine lines, fold and wrinkles, sagging and relaxing of the cutaneous tissue. In this process, the elastic fibers become loser and fibroblast (dermic cells) becomes less active. Due to oxydative stress (free radicals) and lack of normal hydration, keratinization appears and the cutaneous vascular network diminishes.

Histologically, photoageing manifests with a thickening of the epidermis and significant remodelling of the dermal extracellular matrix (ECM), which is thought to underlie clinical features such as wrinkles and loss of elastic recoil. During photoageing the three major classes of dermal ECM components—fibrillar collagens, elastic fibres and glycosaminoglycan—are differentially remodelled, leading to changes in their relative molecular composition, architecture and hence function.

In addition, temporary or even long lasting changes to the skin can occur with age, such as, for example, hormone-associated acne, greasy or dry skin, keratoses, rosacea, light sensitivity or inflammatory erythema.

In vitro and in vivo studies have shown the effective role of antioxidants in the prevention or suppression of disorders such as oxidative damage to DNA, proteins and lipids, enzymes, and several biomolecules. Various scientific studies suggest that a cocktail of antioxidants, presenting different mechanisms of action and molecular structure, is more effective than a single antioxidant due to synergic effects between molecules.

Reactive oxygen species (ROS) are essential for biological functions, such as the respiratory chain, signal transfer and immune response. However, if the ROS exceed a certain level, negative effects can occur. The influence of ROS on premature skin aging and tumor incidence is well-known [Chen, L.; Hu, J. Y.; Wang, S. Q. “The role of antioxidants in photoprotection: A critical review”. J. Am. Acad. Dermatol. 2012, 67:1013-1024].

The skin is subject to ROS and other free radicals on a daily basis. Related damaging factors are, e.g, solar radiation, smoking, alcohol consumption, physical and psychological stress, all of which lead to the formation of ROS and other free radicals in the skin—known as oxidative stress. Oxidative stress promotes the damage of cell components by ROS such as hydrogen peroxide (H₂O₂), hydroxyl radicals (OH.) or superoxide radicals (O₂.).

Antioxidants such as free radical scavengers have the ability to protect cells against the consequences of oxidative stress. These compounds present a mechanism of returning to an active state immediately after neutralization of free radicals. This ensures an abrupt end to the harmful radical chain reaction in the body. When enough antioxidants are available, the cells remain protected. An imbalance between the body's antioxidant defense system and the reactive metabolites induces several physiological processes within the body which can lead to, e.g., cell aging, tissue damage or cancer. If a strong interference between ROS and cells exists, programmed cell death (apoptosis) can be initiated.

Acute exposure of human skin to UV radiation increases the release of proinflammatory mediators from a variety of skin cells, resulting in matrix-metalloproteinase (MMP) and NF-kB signaling. NF-kB (nuclear factor kappa-light-chain-enhancer of activated B cells) is a protein complex that controls transcription of DNA, cytokine production and cell survival. NF-κB is found in almost all animal cell types and it is involved in cellular responses to stimuli such as stress, cytokines, free radicals, UV, and bacterial or viral antigen.

As a major cellular defense pathway, the Nrf2 pathway is known to regulate expression of enzymes involved in detoxification and anti-oxidative stress response. Nrf2 forms heterodimers with small Maf proteins and binds to the antioxidant response elements of target genes when cells are exposed to oxidative stress or electrophiles. Keap1 (Kelch-like ECH-associated protein 1) inhibits the function of Nrf2 by retaining Nrf2 in the cytoplasm under normal physiological conditions, and by allowing nuclear translocation of Nrf2 under stress conditions.

The NF-kB and Nrf2 pathways interface at several points to control the transcription or function of their downstream targets. Antagonism and synergy occur between members of these two pathways through direct effects on transcription factors, protein-protein interactions, or second-messenger effects on target genes. Increasing evidence confirms a crosstalk between Nrf2 and NF-kB under pathological conditions (See for example Bellezza et al. “Nrf2 and NF-κB and their concerted modulation in cancer pathogenesis and progression”. Cancers, 2010, 2:483-497; and Zazueta & Buelna-Chontal “Redox activation of Nrf2 & NF-κB: A double end sword?” Cellular Signalling, 2013, 25:2548-2557). Recent data have suggested the Keap1/CuI3/Rbx1 E3-ubiquitin ligase complex as a commonly machinery regulating both the Nrf2 and the NFkB pathways. Genetic disruption of this complex has been shown to be a key mechanism of NF-kB activation in human lung cancer. In fact, Keap1 functions as an IKKβ E3 ubiquitin ligase. Deletion of Keap1 leads to the accumulation and stabilization of IKKβ and upregulation of NF-kB-derived tumor angiogenic factors.

On one hand, Nrf2 and NF-kB can be functionally antagonistic. Absence of Nrf2 induces more aggressive inflammation through activation of NF-kB and downstream proinflammatory cytokines in astrocytes. Keap1 physically associates with NF-kB-p65 in vitro and in vivo, and NF-kB signaling inhibits Nrf2 pathway through the interaction of p65 and Keap1.

Since it is well accepted that oxidative free-radical stress is an important contributor to the aging process and to many age-related diseases, the focus must be on reducing in vivo oxidative stress. This can be achieved by limiting exposure to outside oxidative agents, such as cigarette smoke and other environmental exposures, such as UV sunlight etc. This is important because oxidative damage apparently increases with age and thus may overwhelm the natural repair systems in the elderly. The best way to neutralize free-radical mediated oxidative stress in the elderly is to ingest diets rich in phytochemicals with antioxidant properties or to take dietary supplements of antioxidants. These are widely found in nature, especially in plant products, and are an extremely diversified group of chemicals. One such naturally occurring antioxidant is garlic.

Garlic (Allium sativum) is one of the best researched herbal remedies and has been commonly also used for treating various health problems for centuries. Garlic is a genus of some 500 species belonging to the family Liliaceae and Allium class of bulbshaped plants. Garlic stimulates the proliferation of macrophages and lymphocytes and protects against the suppression of immunity by ultraviolet radiation.

Garlic has several effects, that is, it acts as an antioxidant, inhibits NF-kB and protects against UV-induced immunity suppression. Its constituents include enzymes (for example, alliinase), sulfur containing compounds such as alliin, and compounds produced enzymatically from alliin (for example, allicin). Other constituents such as arginine, oligosaccharides, flavonoids, and selenium are also available in garlic. S-allylcysteine (SAC) and Sallylmercaptocysteine (SAMC) are the major organosulfur compounds in aged garlic extract (AGE) which prevent oxidant damage. AGE exhibits antioxidant action by scavenging reactive oxygen species (ROS), enhancing cellular antioxidant enzymes such as superoxide dismutase, catalase, glutathione peroxidase and increasing glutathione in cells. AGE protects DNA against free radicals and defends against UV-induced damage. It also protects against some forms of UV-induced immunosuppression and wrinkle formation caused by degradation of collagen fibrils and gelatin fibers.

S-allylcysteine (SAC) is a potent antioxidant agent, a water soluble compound less toxic than other antioxidants, easily absorbed in the gastrointestinal tract, and rapidly detected in several tissues (kidney, liver, lung, brain).

S-allylcysteine has the following formula:

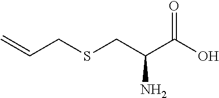

Several studies have been performed in order to test the antioxidant properties of S-allylcysteine (SAC). In these studies, different antioxidant mechanisms have been reported, such as ability to (1) scavenge reactive oxygen (ROS) and nitrogen (RNS) species; (2) increase enzymatic and nonenzymatic antioxidants levels; (3) activate Nrf2 factor; or (4) inhibit some prooxidant enzymes (xanthine oxidase, cyclooxygenase, and NADPH oxidase).

Although aging cannot be effectively halted, perception of facial aging can be altered by reducing its visible signs. Antiaging skin care formulations enable the subject to age well and thus support his self-esteem.

There still remains therefore a need to identify compounds with improved stability and percutaneous absorption, well tolerated, which delivers antiaging benefits to address consumers desire to improve aging conditions while enhancing well-being and avoiding known adverse effects of certain compounds.

In view of this, the present invention discloses new derivatives of S-allylcysteine with surprisingly improved antioxidant, antiaging and antiinflammatory activity, optionally in synergetic combination with other antioxidant compounds.

### SUMMARY OF THE INVENTION

As previously mentioned, the present invention generally relates to novel S-allylcysteine (SAC) derivatives having antioxidant activity.

In a preferred embodiment, at least one of the novel S-allylcysteine (SAC) derivatives is combined with dermatologically acceptable excipients in an antiaging and/or antioxidant formulation for topical administration.

In an alternative preferred embodiment, the novel S-allylcysteine (SAC) derivatives are combined with at least one further active compound and dermatologically acceptable excipients in an antiaging and/or antioxidant formulation for topical administration.

Preferably, in the embodiments wherein there are two or more active compounds, the interaction between the same is synergetic, although in some cases it can be additive.

The inventors have surprisingly identified that these derivatives have activity that mimics the action of SAC and have additional antiaging, antioxidant and antiinflammatory properties according to the moieties used for the preparation thereof.

In a particularly preferred embodiment, the S-allylcysteine (SAC) derivatives disclosed by the invention are esters or/and amides of SAC. Compounds of the present invention include, but are not limited to, derivatives of S-allylcysteine with resveratrol or other flavonoids (such as naringenin, catechins, curcumin, lutein, zeaxantin, quercetin, etc), derivatives of cinnamic acid (such as ferulic acid, caffeic acid, coumaric acid), pantothenic acid, folic acid, hyaluronic acid, pyroglutammic acid, gluconic acid, omega 3 fatty acids (such as docosahexaenoic acid), ascorbic acid and ascorbyl palmitate, tocopherol, hydroxyl acids (such as glycolic acid, lactic acid, citric acid, mandelic acid), butylhydroxyanisole (BHA) and butylhydroxytolune (BHT), gallic acid (such as dodecyl gallate and propyl gallate), terpenoids (such as celastrol, menthol and thymol).

Preferred compounds are esters of SAC with resveratrol and amides of SAC with pyroglutammic acid.

In another embodiment of the present invention, topical formulations comprising said compounds for cosmetic and dermatological use are provided. These compounds have been found to easily penetrate the skin without irritation.

In one preferred aspect of the invention, a compound of the present invention may be used to inhibit matrix metalloproteinase (MMP) expression, which can be induced by UV-exposure and by inflammatory processes in the skin. Accordingly, these compounds can be used to prevent or alleviate the formation of skin wrinkles.

### BRIEF DESCRIPTION OF THE DRAWINGS

FIG. 1—H NMR spectra of (E)-1,1′-((5-(4-((3-(allylthio)-2-ammoniopropanoyl)oxy)styryl)-1,3-phenylene)bis(oxy))bis(3-(allylthio)-1-oxopropan-2-aminium) chloride (Example 1).

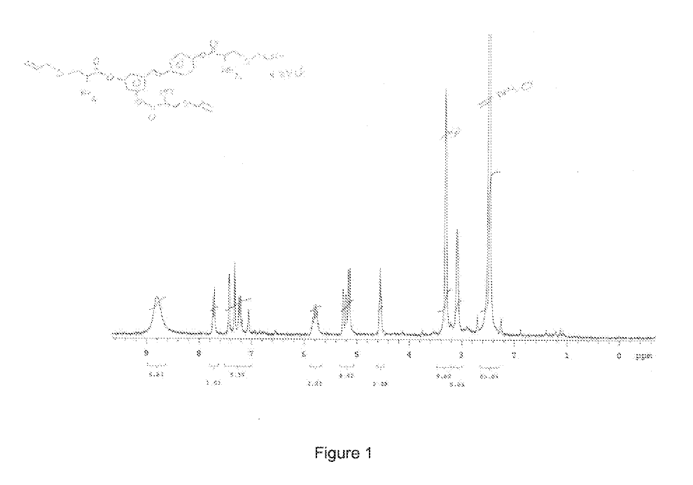

### DESCRIPTION OF THE INVENTION

The present invention provides novel derivatives of S-allylcysteine with antiaging, anti-inflammatory and antioxidant properties.

In one preferred embodiment, said derivatives are esters formed from the carboxylic of SAC, showing general formula:

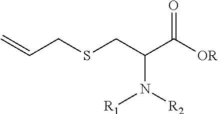

or a pharmaceutically acceptable salt or derivatives thereof
wherein R can be an unsubstituted, branched or unbranched, unsaturated or saturated C1-C6 alkyl, allyl, or aryl group ester,

### and R1=R2=H

Preferably, R can be an antioxidant compound such as: flavonoid or a derivative thereof such as resveratrol (mono- di-, tri-substituted on the oxydryl functions), naringenin, catechins, quercetin, derivatives of gallic acid (such as catechins dodecyl gallate and propyl gallate), or tocopherol or derivatives thereof including the synthetic analogues butylhydroxyanisole (BHA) and butylhydroxytolune (BHT), or carotenoids compounds such as curcumin, or vitamin C or derivatives thereof such as ascorbyl palmitate, or triterpenoid such as celastrol.

In other preferred embodiment, said derivatives are amides formed from amino groups of SAC or a pharmaceutically acceptable salt or derivatives thereof:

Wherein R is hydrogen or an unsubstituted, branched or unbranched, unsaturated or saturated C1-C6 alkyl, allyl, or aryl group, or ions such as alkaline ions or alkaline earth ions (Na⁺, K⁺, Ca⁺⁺, Mg⁺⁺);

and R1≠R2 wherein R1 or R2 is H or an acyl group, wherein said acyl group is linked to an unsubstituted, branched or unbranched, unsaturated or saturated C1-C6 alkyl, allyl, or aryl group, and when R1 different from H then R2 is H

Preferably, R1 or R2 is H or derivatives of pantothenic acid, folic acid, hyaluronic acid, pyroglutammic acid, gluconic acid, omega 3 fatty acids (such as docosahexaenoic acid), vitamin C or derivatives thereof such as ascorbyl palmitate, hydroxyacids (such as glycolic acid, lactic acid, citric acid, mandelic acid)

Resveratrol is a natural phenol produced by several plants in response to injury or when the plant is attacked by pathogens such as bacteria, fungi etc.

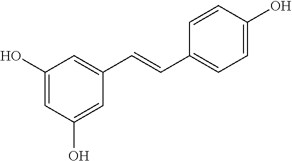 [SMILES: Oc1ccc(/C=C/c2cc(O)cc(O)c2)cc1]

Naringenin is a flavanone compound widely found in citrus fruit (Citrus var.) such as, but not limited to, grapefruit (Citrus paradisi), orange (Citrus sinensis); and also tomato (Solanum lycopersicum).

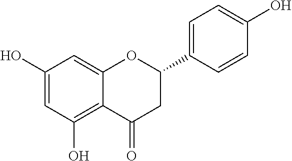 [SMILES: O=C1C[C@@H](c2ccc(O)cc2)Oc2cc(O)cc(O)c21]

Catechin and epicatechin gallate are flavonoids present in tea and other food. They are polyphenos with powerful antioxidant activity.

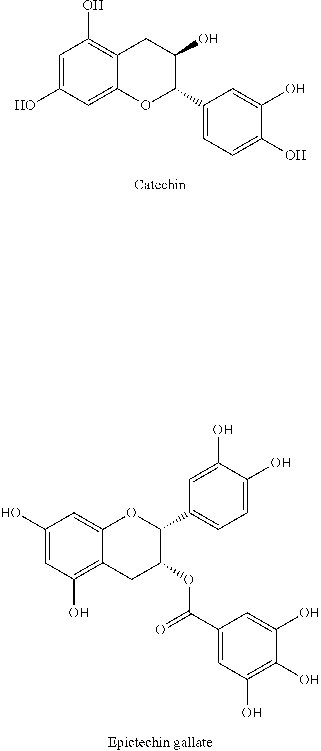 [SMILES: Oc1cc(O)c2c(c1)O[C@@H](c1ccc(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

Curcumin is a natural phenol with antioxidant properties

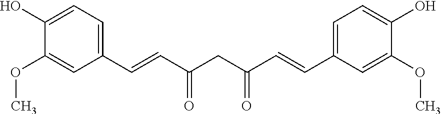 [SMILES: COc1cc(/C=C/C(=O)CC(=O)/C=C/c2ccc(O)c(OC)c2)ccc1O]

Cinnamic acid derivatives are naturally occurring substances found in fruits, vegetables, flowers and are consumed as dietary phenolic compounds

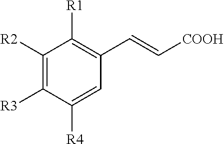

wherein when R1=R2=R3=R4=H, the compound is ciannamic acid;
when R1=OH, the compound is o-coumaric acid;
when R3=OH, the compound is p-coumaric acid;
when R2=R3=OH, the compound is caffeic acid;
when R2=OCH₃ and R3=OH, the compound is ferulic acid.

Caffeic acid and ferulic acid are preferred among the cinnamic acid derivatives.

Pantothenic acid is a water soluble vitamin essential for Coenzyme A synthesis with enhanced suppression of free radical formation in skin fibroblasts

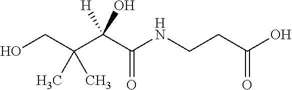

Pyroglutammic acid is (also known as PCA, 5-oxoproline) is an aminoacid derivative in which the free amino group of glutamic acid cyclizes. It is an important component of the Natural Moisturizing Factor (NMF) with excellent humectant effects on the skin.

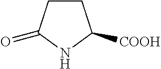

Folic acid is a B complex vitamin which is vital for the formation of red blood cells. It is present in many food and vegetables. The human body needs folic acid to synthesize DNA, repair DNA and it is a cofactor for many biological reactions. It has also free radical scavenging properties and antioxidant activity.

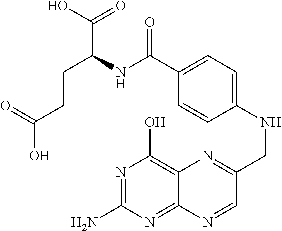 [SMILES: Nc1nc(O)c2nc(CNc3ccc(C(=O)N[C@@H](CCC(=O)O)C(=O)O)cc3)cnc2n1]

Hyaluronic acid is an important component of articular cartilage. It is also a major component of skin, where it is involved in tissue repair.

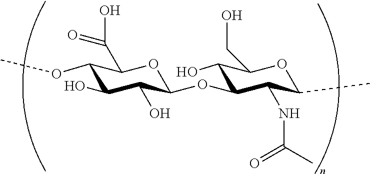

Gluconic acid occurs naturally in fruit, honey and wine. Gluconic acid, and its derivative gluconalactone, are used in many cosmetic preparation as skin conditioning agents.

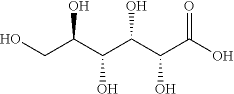 [SMILES: O=C(O)[C@H](O)[C@@H](O)[C@H](O)[C@H](O)CO]

Docosahexaenoic acid is an omega-3 fatty acid and is contained in fish oils, chloroplast containing microalgae and cyanobacteria, like spirulina.

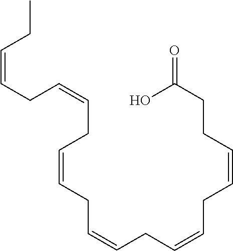 [SMILES: CC/C=C\C/C=C\C/C=C\C/C=C\C/C=C\C/C=C\CCC(=O)O]

Ascorbic acid, also known as vitamin C, and its derivative ascorbyl palmitate, are essential nutrients for man. They are widely used to prevent oxidation in many preparations

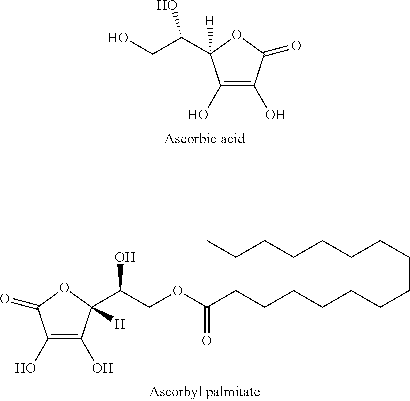 [SMILES: O=C1O[C@H]([C@@H](O)CO)C(O)=C1O ; CCCCCCCCCCCCCCCC(=O)OC[C@H](O)[C@H]1OC(=O)C(O)=C1O]

Tocopherol, also known as vitamin E, is a fat-soluble antioxidant present in the skin and presents many other functions in the body.

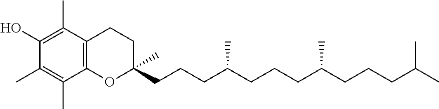 [SMILES: Cc1c(C)c2c(c(C)c1O)CC[C@@](C)(CCC[C@H](C)CCC[C@H](C)CCCC(C)C)O2]

Hydroxyacids, or alpha hydroxy acids (AHAs), are a class of chemical compounds that consists of a carboxylix acid substituted with a hydroxyl group on the adjacent carbon, widely used in cosmetic field. The preferred hydroxyacids are glycolic acid (R=H), lactic acid (R=CH₃), citric acid (R=—(CH₂—COOH)₂) and mandelic acid (R=—C₆H₆).

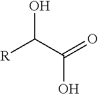

Butyl hydroxyanisole (BHA) and butylhydroxytoluene (BHT) are synthetic antioxidants commonly used in cosmetics.

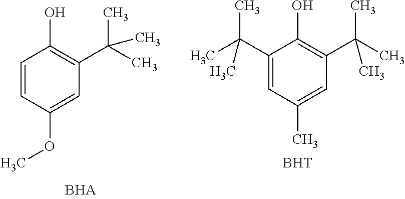

Derivatives of gallic acid such dodecyl gallate, propyl gallate are also commonly used:

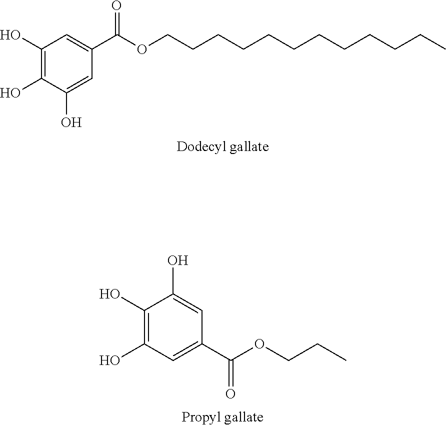 [SMILES: CCCCCCCCCCCCOC(=O)c1cc(O)c(O)c(O)c1 ; CCCOC(=O)c1cc(O)c(O)c(O)c1]

Celastrol is a chemical compound isolated from the root extracts of Tripterygium wilfordii (Thunder god vine) and Celastrus regelii. Celastrol is a pentacyclic triterpenoid with antioxidant and anti-inflammatory properties.

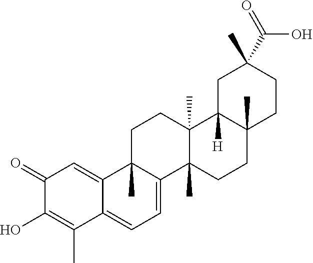

The inventors have now surprisingly identified improved antioxidant, antiinflammatory and antiaging properties in structures derived from the reaction of the compounds defined above with S-allylcysteine.

According to an embodiment of the invention, the novel S-allylcysteine (SAC) derivatives, namely S-allylcysteine esters and amides, of the present invention can be prepared by following the steps below:
(a) Solubilize the (S)-allylcysteine derivative;
(b) Add the alcoholic, or carboxylic compound for the preparation of the ester or amide;
(c) Add the coupling agents; and
(d) Purify the resulting ester or amide by chromatography or crystallization

The novel S-allylcysteine (SAC) derivatives of the present invention can be prepared following the general schemes below:

A) Esters of S-Allylcysteine

The esters of S-allylcysteine were prepared according to the following procedure. N-((tert-Butoxycarbonyl)-S-allylcysteine, DCC, DMAP and HOBt were added to a solution of the alcohol or the phenolic compound selected for the preparation of the ester. When the reaction was completed the dicyclohexylurea was removed and the product was purified. Then, tert-butoxycarbonyl group was removed by treating the product with HCl in dioxane.

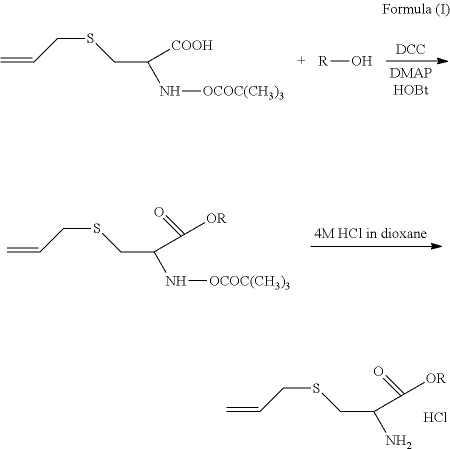

B) Amides of S-Allylcysteine

The amides of S-allylcysteine were prepared according to the following procedures. (i) S-Allylcysteine methylester hydrochloride, obtained by reacting N-Boc-cysteine methyl ester (acquired from Sigma-Aldrich) with allylbromide, was added (ii) to a solution of the carboxylic acid in presence of EDAC, DMAP and HOBt or in alternative (iii) to a solution of succinimide ester of the carboxylic compound selected for the preparation of the amide in presence of triethylamine (TEA) or diisopropyilethylamine (DIPEA).

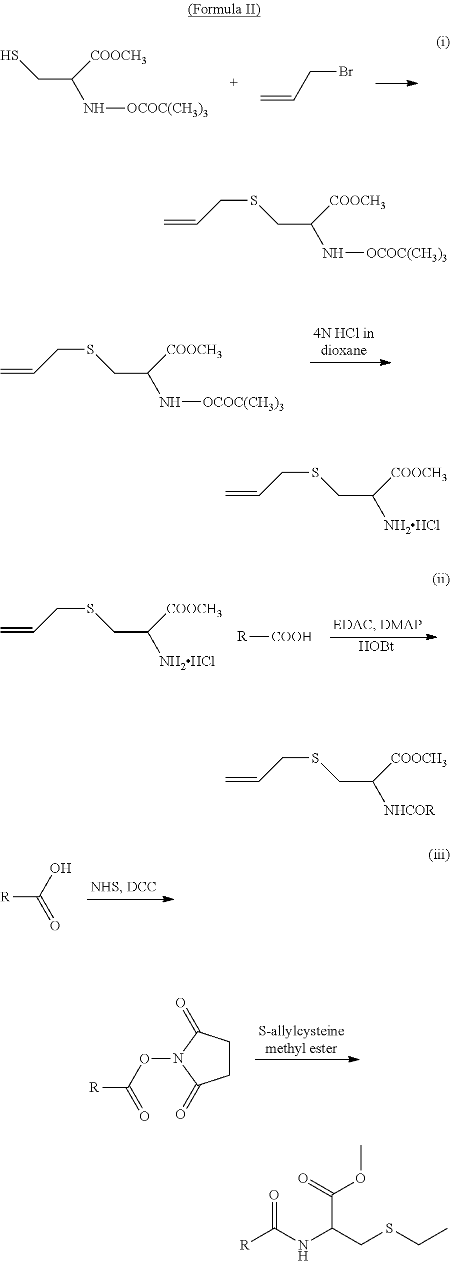

wherein:
DCC=N,N′-dicyclohexylcarbodiimide
DMAP=4-N,N-dimethylaminopyridineHOBt=1-hydroxy benzotriazole
HOBt=1-hydroxy benzotriazole
EDAC=1-Ethyl-3-(3-dimethylamino propyl) carbodiimide hydrochloride
DCU=dicyclohexylurea

### BOC=N-(tert-Butoxycarbonyl)

### NHS=N-hydroxysuccinimide

In one preferred aspect of the invention, a compound selected from the group consisting of: S-allylcysteine derivatives with resveratrol, or other flavonoids (such as naringenin, catechins, curcumin, quercetin, etc), derivatives of cinnamic acid (such as ferulic acid, caffeic acid, coumaric acid), pantothenic acid, folic acid, hyaluronic acid, pyroglutammic acid, gluconic acid, omega 3 fatty acids (such as docosahexaenoic acid), ascorbic acid and ascorbyl palmitate, tocopherol, hydroxyacids (such as glycolic acid, lactic acid, citric acid, mandelic acid), butylhydroxyanisole (BHA) and butylhydroxytoluene (BHT), derivatives of gallic acid (such as dodecyl gallate and propyl gallate), terpenoids such as celastrol, menthol and thymol may be used to inhibit matrix metalloproteinase (MMP) expression, which can be induced by UV-exposure and by inflammatory processes in the skin.

Accordingly, these compounds can be used to prevent or alleviate the formation of skin wrinkles associated with collagen which has been lost or destroyed. The treatable and/or preventable signs of aging which benefit from application of the at least one compound of the present invention include wrinkles, skin with fine lines, wizened skin, lack of skin elasticity, lack of skin tone, thinned skin, dry skin, sagging skin, skin suffering from degradation of collagen fibres, flaccid skin, sagging skin, and skin suffering from internal degradation.

In particular, such compounds may be used to protect against and/or alleviate signs of aging by acting on at least one sign of skin aging or at least one sign of a skin damage condition associated with aging, wherein the sign of skin aging or skin damage is present on skin of the face, body or the scalp of a subject. These compounds have been advantageously found to be non-irritating to the skin. Accordingly, application to the skin can be made in a substantially pure form thereof or diluted in appropriate vehicles with optional dermatologically acceptable excipients.

The skilled person will appreciate that each one of these S-allylcystein compounds may be used alone or in any combination with one or more of the compounds disclosed above or other compounds with known antioxidant and/or antiinflammatory activity to provide effects on at least one sign of aging.

Preferably, the compounds of the invention may be presented in the form of a skin-care product. Preferably, the compounds are comprised in a topical composition. Said composition may be in the form of a gel, cream, milk, lotion, serum, oil (for example massage oil), scrub, powder, mask, toner, or the like. Further preferred forms of the composition include a soap or a cleanser (such as a facial cleanser), a shampoo, a shower or bath gel.

In an alternative embodiment of the invention, the composition is in the form of a colour cosmetic product such as a foundation, a base for make-up, a concealer, pressed powder, mascara or a lipstick. It may also be incorporated into a wrap or film, a mask, a patch, a cloth or blanket, a pad, a sheet, a wipe, a pen or the like. Most preferably the product is a leave-on topical product, that is, a product to be applied to the skin without a deliberate rinsing step soon after its application to the skin.

Examples of “cosmetically acceptable compounds” are selected from the group consisting of: silicones, emulsifiers, surfactants, thickeners, powders, film formers, rheology modifying agents, propellants, fragrance, opacifiers, preservatives, colorants, pigments, buffers, chelating agents, sensory enhancers and combinations thereof. It will be appreciated by those skilled in the art, however, that compositions from the present invention may comprise further suitable cosmetically acceptable compounds.

The compositions of the invention may further comprise skin-care actives and dermatologically acceptable compound such as UV filters (such as ethylhexyl methoxycinnamate, octocrylene, ethylhexyl salicylate, butyl methoxydibenzoylmethane, titanium dioxide or phenylbenzimidazol sulfonic acid, for example), skin purifying actives, peeling agents, sebum reducing agents, mattifying agents, anti-perspirant actives, self-tanning actives, skin plumping actives, barrier function enhancing agents, surfactants and other cleansing agents, delivery enhancers and the like. The term “dermatologically acceptable compounds” as used herein means that the carriers described are suitable for use in contact with mammalian keratinous tissue without causing any adverse effects such as toxicity, incompatibility, instability and allergic response.

The compositions of the invention may also comprise a vehicle which may be formulated to improve the delivery of the actives to the skin. The topical composition according to the present invention may be prepared in a manner well known in the art of preparing skin care products. The active components are generally incorporated in a dermatologically acceptable vehicle or carrier. The active components can suitably first be dissolved or dispersed in a portion of the water or another solvent or liquid to be incorporated in the composition.

The composition may be in the form of an emulsion, such as the oil-in-water, water-in-oil, silicone-in-water, water-in-silicone types, or a multiple emulsion such as a triple emulsion (for example water/oil/water (W/O/W) emulsion), phase inversion temperature (P.I.T.) emulsion, phase inversion concentration (P.I.C.) emulsion, wax-in-water emulsion, microemulsion or D-phase gel or the like. Compositions may also be in the form of a cream, gel, a solution, a dispersion (for example a hydro-dispersion or lipo-dispersion), a paste or a solid (for example, a solid stick, pressed powder). Alternatively, the compositions may be in the form of an alcohol-based system or an aerosol.

In a preferred embodiment, the composition is in the form of an emulsion. In a further preferred embodiment, the composition is in the form of an oil-in-water emulsion or a water-in-oil emulsion.

The composition may be packaged in any suitable manner such as a jar, a bottle, a tube, a pump, a pump dispenser-tube, an aerosol or foam dispensing pump, a roll-ball, a stick, a brush or a sachet, for example. It may also be incorporated in a capsule, an ampoule or a dropper system.

In a preferred embodiment of the invention, the total concentration of active compounds, including at least one S-allylcysteine derivative from the present invention and optional active compounds from the art, in the cosmetic or dermatological composition of the invention is in the range of about 0.001-50% w/w. In a further preferred embodiment, the total concentration of active compounds is in the range 0.01-10% w/w. In a still further preferred embodiment, the total concentration of active compounds is in the range 0.05-5% w/w.

In a preferred embodiment of the invention, the concentration of at least one cosmetically acceptable excipient in the cosmetic or dermatological composition of the invention is in the range of about 50-99.999% w/w. In a further preferred embodiment, at least one active is in the range 0.01-10% w/w. In a still further preferred embodiment, at least one active is in the range 95-99.95% w/w.

According to the present invention, the terms “cosmetically acceptable compounds”, “cosmetically acceptable excipients”, “dermatologically acceptable compounds” and “dermatologically acceptable excipients” and used interchangeably herein and refer to compounds that are commonly used in dermatologic and cosmetic formulations combined with the active ingredients for application to the skin, body and/or scalp. Particularly, they refer to compounds which render, without limitation, shape, flavor, stability and color to the final composition, in a safe and tolerable manner for the user. In some embodiments a “cosmetically acceptable” component can facilitate absorption of one or more active ingredients in application.

The terms “S-allylcysteine derivatives”, “S-allylcysteine hybrids” and “derivatives”, as defined herein, are used interchangeably and refer to compounds that are derived from S-allylcysteine.

The terms “active compound” and “active compounds”, as used herein, refer to compounds which, upon topical administration, provide a desired cosmetic and/or dermatological effect to the subject. Particularly, said “active compounds” are S-allylcysteine derivatives, unless otherwise indicated.

The present invention will now be illustrated by the following examples. It is understood, however, that such examples are provided for illustration purposes only, and the invention is not intended to be limited thereby.

### Example 1. Preparation of (E)-1,1′-((5-(4-((3-(allylthio)-2-ammoniopropanoyl)oxy)styryl)-1,3-phenylene)bis(oxy))bis(3-(allylthio)-1-oxopropan-2-aminium) Chloride (S-allylcysteine Resveratrol Ester)

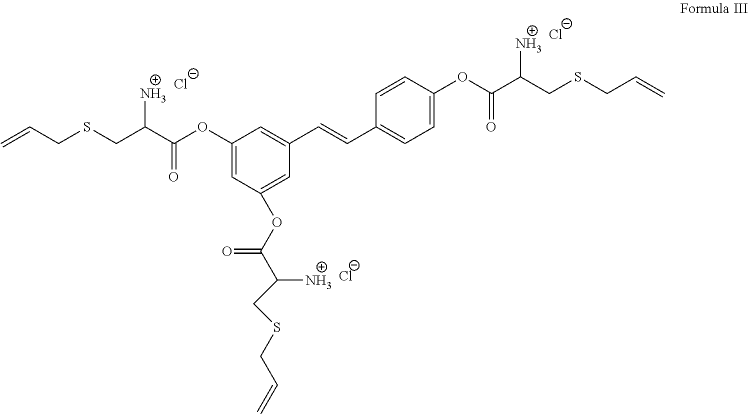 [SMILES: [Cl-] ; C=CCSCC([NH3+])C(=O)Oc1ccc(/C=C/c2cc(OC(=O)C([NH3+])CSCC=C)cc(OC(=O)C([NH3+])CSCC=C)c2)cc1 ; [Cl-] ; [Cl-]]

To a solution of resveratrol (115 mg, 0.504 mmol) in anhydrous tetrahydrofurane (THF), HOBt (340.5 mg, 2.52 mmol), DCC (519.2 mg, 2.52 mmol), DMAP (30.8 mg, 0.252 mmol) were added at 0° C. and stirred for 20 min. A solution of N-BOC-S-allylcysteine (659 mg, 2.52 mmol) in THF (5 ml) was added, and the mixture was allowed to warm to room temperature and stirred for 24 h. DCU was separated by filtration and the solution was evaporated to dryness under reduced pressure. The residue was dissolved in CH₂Cl₂ and the organic solution was washed successively with 5% NaHCO₃, water, 1N HCl and brine and finally dried (Na₂SO₄) and evaporated under reduced pressure to give a crude product which was purified by flash chromatography on silica using 0.6% CH₃OH in CH₂Cl₂ to yield a white solid.

In a round bottom flask, cooled with an ice bath, 0.4 ml of 4N HCl in dioxane and 25 μl of water (as a scavenger) were added to a solution of triester of resveratrol with N—BOC-S-allylcysteine (45 mg, 0.047 mmol) in 100 μl of dioxane. The resultant solution was stirred at room temperature for 6 h and then was evaporated under reduced pressure to dryness to yield a white solid, which was washed with ether. M.p. 166.6-170.2° C. (dec.).

¹H NMR (DMSO-d₆) δ: 8.90 (s, 9H), 7.70 (d, 2H), 5.50-7.04 (m, 7H), 5.90-5.70 (m, 3H), 5.30-5.08 (m, 6H), 4.55 (s, 3H), 3.30 (s, 6H), 3.08 (s, 6H).

### Example 2. Preparation of L-Pyroglutamyl-(R)-3-Allylsulfanyl-2-amino-propionic Acid Methyl Ester

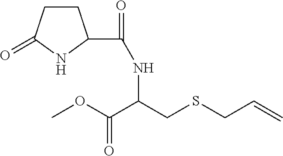 [SMILES: C=CCSCC(NC(=O)C1CCC(=O)N1)C(=O)OC]

### Formula IV

The synthesis of the compound is performed in four steps.

(i) (R)-3-Allylsulfanyl-2-tert-butoxycarbonylaminopropionic Acid Methyl Ester.
N-BOC-cysteine methyl ester (obtained from Sigma-Aldrich, Milan, Italy) (1.00 g, 4.25 mmol) was dissolved in anhydrous dichloromethane (6 mL) under an atmosphere of argon. After cooling the solution at 0° C., triethylamine (0.65 mL, 4.675 mmol) and allyl bromide (0.4 mL, 4.675 mmol) were added. The reaction mixture was stirred at rt for 18 h before being concentrated in vacuo. The residue was partitioned between EtOAc and 1M HCl. The organic phase was washed with brine, dried (Na₂SO₄), filtered and concentrated under reduced pressure. The crude material was purified by flash chromatography on silica using 7% EtOAc in Cyclohexane to yield a white solid.

(ii) (R)-3-Allylsulfanyl-2-aminopropionic Acid Methyl Ester Hydrochloride.
In a round bottom flask, cooled with an ice bath, (R)-3-Allylsulfanyl-2-tert-butoxycarbonylaminopropionic acid methyl ester (300 mg, 1.09 mmol) was dissolved in 4M hydrogen chloride in 1,4-dioxane (3.75 ml) and 0.30 mL of water (as a scavenger). The resultant solution was stirred at rt for 35 min and then was evaporated in vacuo to dryness to yield a white solid, which was washed with ether.

(iii) L-Pyroglutamic Acid Succinimide Ester (L-pGlu-OSu).
L-Pyroglutamic acid (1.00 g, 7.75 mmol) and N-hydroxysuccinimide (1.07 g, 9.3 mmol) were dissolved on heating in dioxane (18 ml) and after cooling to room temperature, dicyclohexylcarbodiimide (DCC, 1.597 mg, 7.75 mmol) was added to the solution. The mixture was stirred at room temperature for 18 h and then the DCU was separated by filtration. After evaporation of the solvent, an oil was obtained which was crystallized from dichloromethane.

(iv) L-Pyroglutamyl-L-S-allylcysteine Methyl Ester.
A solution of (iii) L-Pyroglutamic acid succinimido ester (94 mg, 0.4158 mmol) in anhydrous THF (2 ml) was added to a solution of (i) (R)-3-allylsulfanyl-2-aminopropionic acid methyl ester hydrochloride (80 mg; 0.378 mmol) and triethylamine (42 mg; 0.4158 mmol) in 2 ml of anhydrous THF and the mixture was stirred at room temperature. After 6 h, the reaction mixture was filtered and the organic solution was evaporated under reduced pressure. The residual pale yellow liquid was dissolved in CH₂Cl₂ and the solution was successively washed with 10% citric acid, water, 5% NaHCO₃, brine and finally dried (Na₂SO₄) and evaporated under reduced pressure to give a crude product as a thick yellow liquid, which crystallized after the addition of few drops of ether. M.p. 44.8-48.5° C.

¹H NMR (CDCl₃) δ: 6.90 (br s, 1H, collapsed with D₂O), 6.20 (d, 1H, collapsed with D₂O), 5.82-5.66 (m, 1H), 5.20-5.08 (t, 2H), 4.85-4.75 (m, 1H), 4.15-4.25 (m, 1H), 3.78 (s, 3H), 3.18-3.05 (m, 2H), 3.02-2.95 (dd, 1H), 2.90-2.75 (dd, 2H), 2.60-2.15 (m, 4H).

### Example 3: Preparation of 3-(allylthio)-1-methoxy-1-oxopropan-2-aminium Chloride

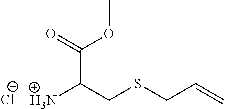

### Formula V

(i) Preparation of methyl 3-(allylthio)-2-((tert-butoxycarbonyl)amino)propanoate
N-tert-Butoxycarbonyl)-L-cysteine methyl ester (1.66 ml; 8.07 mmol) was dissolved in anhydrous CH₂Cl₂ (11 ml) in a 50 ml double neck round bottom flask kept at 0° C. After that allylbromide (1.24 ml; 8.87 mmol) and TEA (1.24 ml; 8.87 mmol) were added, the reaction mixture was allowed to warm to room temperature and it was stirred under nitrogen for 18 hours. At the end of the reaction, the salts were filtered and the solvent evaporated. The residue was treated with ethyl acetate and washed five times with a cold solution of 1N HCl, and once with cold brine. The organic phase was dryed on sodium sulphate and the solvent evaporated.

A white solid having melting point 41.2-42.6° C. was obtained.

(ii) Preparation of 3-(allylthio)-1-methoxy-1-oxopropan-2-aminium Chloride

Methyl 3-(allylthio)-2-((tert-butoxycarbonyl)amino)propanoate (1.93 g; 7.00 mmol), prepared as in point (i), was dissolved in 1.4 ml of water and 20 ml of 3M HCl in dioxane in a 50 ml round bottom flask. The reaction mixture was stirred at room temperature, under nitrogen, for 35 minutes. At the end of the reaction, the solvent was evaporated and the residue was crystallyzed by ethyl ether.

A white solid was obtained with a melting point of 116.3-118.4° C.

### Example 4: Preparation of methyl (E)-S-allyl-N-(3-(3,4-dihydroxyphenyl)acryloyl) Cysteinate

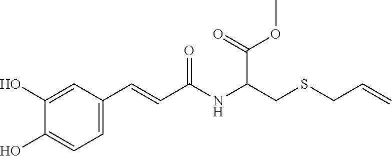 [SMILES: C=CCSCC(NC(=O)/C=C/c1ccc(O)c(O)c1)C(=O)OC]

### Formula VI

Caffeic acid (100 mg; 0.55 mmol) was dissolved in anhydrous THF (2.5 ml) in a 25 ml double neck round bottom flask, cooled on ice bath at 0° C. Then, 3-(allylthio)-1-methoxy-1-oxopropan-2-aminium chloride (117 mg; 0.55 mmol), HOBt (83 mg; 0.61 mmol) and TEA (0.232 ml; 1.66 mmol) were added. After 10 minutes, EDC-HCl (106 mg; 0.55 mmol), previously dissolved in THF (1.5 ml), was added and the reaction mixture was stirred at room temperature, under nitrogen, for 18 hours. At the end of the reaction, the salts are filtered and the solvent evaporated. The residual oil is treated with ethyl acetate and washed three times with a cold solution of 1N HCl, twice with 5% NaHCO₃ and once with cold brine. Finally, the organic phase was dryed on anhydrous sodium sulphate and evaporated. The crude residue was purified by flash chromatography on silica, using 1.7% of MeOH in CH₂Cl₂ to yield pure product as a brown viscous oil.

¹H-NMR (300 MHz, DMSO-d₆): δ=9.29 (s, 2H, —OH, collapsed with D₂O), 8.42 (d, 1H, J=7.5 Hz, —NH, collapsed with D₂O), 7.22 (d, 1H, J=15.9 Hz, =CH), 6.95 (s, 1H, ArH), 6.81 (d, 1H, J=8.1 Hz, ArH), 6.71 (d, 1H, J=8.1 Hz, ArH), 6.41 (d, 1H, J=15.6 Hz, =CH), 5.83-5.61 (m, 1H, —CH═CH₂), 5.11 (dd, 2H, J=27.0 e 17.1 Hz, —CH═CH₂), 4.53 (t, 1H, J=6.75 Hz, —CH—NH₂), 3.61 (s, 3H, —OCH₃), 3.18 (d, 2H, J=6.9 Hz, —CH₂), 2.79 (ddd, 2H, J=24.3, 14.1 e 4.8 Hz, —CH₂) ppm.

### Example 5: Preparation of Methyl (E)-S-allyl-N-(3-(4-hydroxy-3-methoxyphenyl) acryloyl)cysteinate

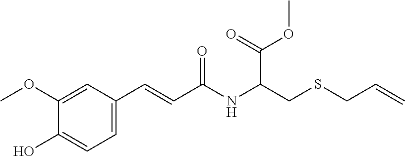 [SMILES: C=CCSCC(NC(=O)/C=C/c1ccc(O)c(OC)c1)C(=O)OC]

### Formula VII

Ferulic acid (275 mg; 1.417 mmol) was dissolved in anhydrous DMF (0.5 ml) in a 25 ml double neck round bottom flask, cooled at 0° C. Then, HOBt (402 mg; 1.56 mmol), EDC-HCl (352 mg; 1.842 mmol), DMAP (17 mg; 0.141 mmol) and a solution of 3-(allylthio)-1-methoxy-1-oxopropan-2-aminium chloride (300 mg; 1.417 mmol) and DIPEA (0.246 ml; 1.417 mmol) in 1 ml of anhydrous DMF were added. The reaction mixture was allowed to warm to room temperature and stirred under nitrogen for 22 h. At the end of the reaction, DMF was evaporated and the residue was taken up with ethyl acetate and washed three times with a cold solution of 1N HCl, twice with 5% NaHCO₃ and once with cold brine. The organic phase was dryed on anhydrous sodium sulphate and purified by flash chromatography on silica, eluting with a gradient of a CH₂Cl₂/MeOH mixture. The obtained colourless oil was crystallized after treatment with an ethyl ether/MeOH solution.

A white crystalline solid with melting point 107.8-108.6° C. was obtained.

¹H-NMR (300 MHz, DMSO-d₆): δ=9.41 (s, 1H, —OH, collapsed with D₂O), 8.21 (d, 1H, J=7.5, —NH, collapsed with D₂O), 7.32 (d, 1H, J=15.9 Hz, =CH), 7.15 (s, 1H, ArH), 6.99 (d, 1H, J=8.0 Hz, ArH), 6.78 (d, 1H, J=8.0 Hz, ArH), 6.57 (d, 1H, J=15.3 Hz, =CH), 5.83-5.62 (m, 1H, —CH═CH₂), 5.11 (dd, 2H, J=27.0 e 17.1 Hz, —CH═CH₂), 4.58 (t, 1H, J=6.75 Hz, —CH—NH₂), 3.79 (s, 3H, Ar—OCH₃), 3.61 (s, 3H, —OCH₃), 3.18 (d, 2H, J=6.9, —CH₂), 2.79 (ddd, 2H, J=24.3, 14.1 e 4.8 Hz, —CH₂) ppm.

### Example 6: Preparation of methyl S-allyl-N-(2-hydroxy-2-phenylacetyl)cysteinate

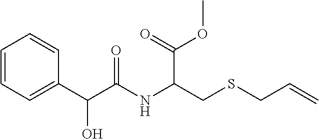 [SMILES: C=CCSCC(NC(=O)C(O)c1ccccc1)C(=O)OC]

### Formula VIII

Mandelic acid (215 mg; 1.417 mmol) was dissolved in 1 ml of anhydrous DMF in a 25 ml double neck round bottom flask, cooled on ice at 0° C. Then, HOBt (211 mg; 1.56 mmol), EDC-HCl (352 mg; 1.842 mmol), DMAP (17 mg; 0.141 mmol) and a solution of 3-(allylthio)-1-methoxy-1-oxopropan-2-aminium chloride (300 mg; 1.417 mmol) and DIPEA (0.246 ml; 1.417 mmol) in 1 ml of anhydrous DMF were added. The reaction mixture was allowed to warm to room temperature and stirred under nitrogen for 22 hours.

At the end of the reaction, DMF was evaporated and the residue was taken up with ethyl acetate and washed three times with a cold solution of 1N HCl, five times with 5% NaHCO₃ and once with cold brine. The organic phase was dryed on anhydrous sodium sulphate and purified by flash chromatography on silica, eluting with a gradient of a CH₂Cl₂/MeOH mixture. A brown viscous oil was obtained.

¹H-NMR (300 MHz, DMSO-d₆): δ=8.30 (d, 1H, J=6.9, —NH, collapsed with D₂O), 7.51-7.20 (m, 5H, ArH, 6.30 (d, 1H, J=4.8 Hz, —OH collapsed with D₂O), 5.79-5.61 (m, 1H, —CH═CH₂), 5.08 (dd, 2H, J=27.0 e 17.1 Hz, —CH═CH₂), 4.98 (d, 1H, J=4.8 Hz, —CH—OH), 4.42 (t, 1H, J=6.75 Hz, —CH—NH₂), 3.60 (s, 3H, —OCH₃), 3.11 (d, 2H, J=6.9, —CH₂), 2.90-2.70 (m, 2H, —CH₂) ppm.

### Example 7: Preparation of methyl S-allyl-N-((2R,4aS,6aS,12b R,14aS)-10-hydroxy-2,4a,6a,9,12b,14a-hexamethyl-11-oxo-1,2,3,4,4a,5,6,6a,11,12b,13,14,14a,14b-tetradecahydropicene-2-carbonyl)cysteinate

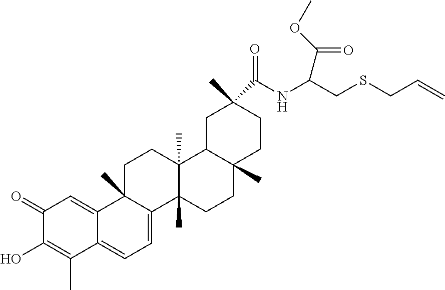 [SMILES: C=CCSCC(NC(=O)[C@]1(C)CC[C@]2(C)CC[C@]3(C)C4=CC=C5C(=CC(=O)C(O)=C5C)[C@]4(C)CC[C@@]3(C)C2C1)C(=O)OC]

### Formula IX

3-(Allylthio)-1-methoxy-1-oxopropan-2-aminium chloride (26 mg; 0.122 mmol) was dissolved in 2 ml of anhydrous THF and 0.2 ml of anhydrous DMF in a 25 ml double neck round bottom flask, cooled on ice at 0° C. Then, DIPEA (23 ml; 0.133 mmol) and celastrol (50 mg; 0.111 mmol), HOBt (15 mg; 0.111 mmol) and EDC-HCl (23.4 mg; 0.122 mmol) were added. The reaction mixture was allowed to warm to room temperature and then was stirred under nitrogen for 5 hours. At the end of the reaction, the solvent was evaporated and the residue was taken up with a CH₂Cl₂ and washed with water. The organic phase was dryed on sodium sulphate and purified by flash chromatography on silica, eluting with a 0.5% MeOH in CH₂Cl₂. An amorphous orange solid was obtained which after treatment with a mixture of ethyl ether/petroleum ether/cyclohexane (1:1:1) crystallized.

An orange crystalline solid with a melting point of 62.8-64.7° C. was obtained.

¹H-NMR (300 MHz, acetone-d₆): δ=7.53 (s, 1H, —OH, collapsed with D₂O), 7.22 (d, 1H, J=7.2 Hz, —NH, slowly collapsed with D₂O), 7.10 (d, 1H, J=7.2 Hz, =CH), 6.41 (d, 2H, J=6.6 Hz, 2=CH), 5.80-5.60 (m, 1H, —CH═CH₂), 5.03 (dd, 2H, J=29.1 e 16.8 Hz, —CH═CH₂), 4.41 (t, 1H, J=6.75 Hz, —CH—NH₂), 3.60 (s, 3H, —OCH₃), 3.10 (d, 2H, J=6.9 Hz, —CH₂), 2.82 (m, 2H, —CH₂), 2.22 (s, 3H, —CH₃), 2.01-1.84 (m, 2H, —CH₂), 1.82-1.58 (m, 2H, —CH₂), 1.59 (s, 3H, —CH₃), 1.38 (s, 3H, —CH₃), 1.21 (s, 3H, —CH₃), 1.18 (s, 3H, —CH₃), 1.00-0.79 (m, 2H, —CH₂), 0.70 (s, 3H, —CH₃) ppm.

### Example 8: Preparation of methyl S-allyl-N-(2-hydroxyacetyl)cysteinate

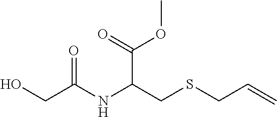

### Formula X

(i) Preparation of 2,5-dioxopyrrolidin-1-yl 2-hydroxyacetate

Glycolic acid (1 g; 13.15 mmol) and N-hydroxysuccinimide (1.54 g; 13.41 mmol) were dissolved in 12 ml of anhydrous CH₃CN in a 50 ml double neck round bottom flask. Then DCC (2.76 g; 13.41 mmol) was added and the reaction mixture was stirred under nitrogen for 4 hours. The DCU formed was filtered and the solvent was evaporated to dryness under reduced pressure. The obtained residue was thoroughly washed with ether, yielding a white solid with melting point 115.9-118.7° C.

(ii) Preparation of methyl S-allyl-N-(2-hydroxyacetyl)cysteinate.

3-(Allytthio)-1-methoxy-1-oxopropan-2-aminium chloride (400 mg; 1.89 mmol) was dissolved in anhydrous DMF (1 ml) in a 25 ml double neck round bottom flask. Then, DIPEA (0.36 ml; 2.07 mmol) and 2,5-dioxopyrrolidin-1-yl 2-hydroxyacetate (358.3 mg; 2.07 mmol), prepared as described above at point (i), were added and the mixture was stirred at room temperature, under nitrogen for 22 hours. At the end of the reaction, the solvent was evaporated and the residue was taken up with CH₂Cl₂ and washed three times with a cold solution of 1N HCl, three times with 5% NaHCO₃ and once with cold brine. The organic phase was dryed on anhydrous sodium sulphate and evaporated.

A yellow oil was obtained.

¹H-NMR (300 MHz, DMSO-d₆): δ=8.1 (d, J=6.8 Hz, 1H, —NH—, collapsed with D₂O), 5.81-5.62 (m, 1H, —CH═CH₂), 5.60 (t, 1H, J=6.7, —OH, collapsed with D₂O), 5.10 (dd, 2H, J=27.0 e 17.1, —CH═CH₂), 4.51 (t, 1H, J=6.75, —CH—), 3.83 (d, 2H, J=6.3, —CH₂), 3.61 (s, 3H, —OCH₃), 3.11 (d, 2H, J=6.9 Hz, —CH₂—), 2.82 (m, 2H, —CH₂) ppm

### Example 9: Preparation of N⁵-(3-(allylthio)-1-methoxy-1-oxopropan-2-yl)-N²-(4-(((2-amino-4-hydroxypteridin-6-yl)methyl)amino)benzoyl)glutamine

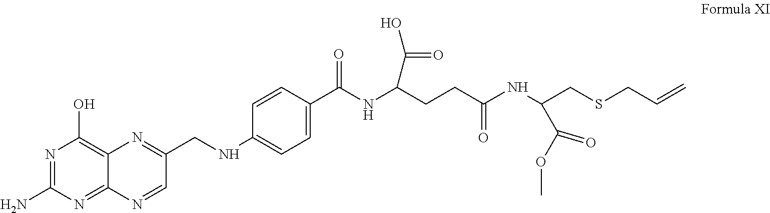 [SMILES: C=CCSCC(NC(=O)CCC(NC(=O)c1ccc(NCc2cnc3nc(N)nc(O)c3n2)cc1)C(=O)O)C(=O)OC]

Folic acid (200 mg; 0.416 mmol) was dissolved in anhydrous DMSO (7.5 ml) in a 50 ml double neck round bottom flask. Then, DCC (129 mg; 0.624 mmol) and NHS (72 mg; 0.624 mmol) were added and the mixture was stirred at room temperature under nitrogen and in the dark for 22 hours. At the end of the reaction, the DCU was filtered and 3-(allytthio)-1-methoxy-1-oxopropan-2-aminium chloride (88 mg; 0.416 mmol) and DIPEA (130 □l; 1.04 mmol) were added and the mixture was stirred at room temperature for 22 hours. At the end, after addition of 100 ml of diethyl ether/acetone (8:2) a precipitate was obtained which was separated by centrifugation and then further washed three times with aceton and three times with diethy ether.

The obtained yellow solid has a melting point of 233.8-236.9° C.

¹H-NMR (300 MHz, DMSO-d₆): δ=11.39 (s, 1H, —OH, collapsed with D₂O), 8.63 (s, 1H, —CH), 8.37 (d, 1H, J=7.2 Hz, —NH, collapsed with D₂O), 8.17 (d, 1H, J=8.1 Hz, —NH, collapsed with D₂O), 8.0 (d, 1H, —NH, collapsed with D₂O), 7.62 (d, 2H, J=8.4 Hz, ArH), 6.92 (s, 2H, —NH₂, collapsed with D₂O), 6.60 (d, 2H, J=8.4 Hz, ArH), 5.70 (m, 1H, —CH═CH₂), 5.09 (dd, 2H, J=29.1 e 16.7 Hz, —CH═CH₂), 4.42 (m, 4H, 2-CH e —CH₂), 3.60 (s, 3H, —OCH₃), 3.10 (d, 2H, J=6.9 Hz, —CH₂), 2.73 (m, 2H, —CH₂), 2.22 (m, 2H, —CH₂), 1.97 (m, 2H, —CH₂) ppm.

### Example 10: Preparation of: 1,1′-((((1 E,3Z6E)-3-hydroxy-5-oxohepta-1,3,6-triene-1,7-diyl)bis(2-methoxy-4,1-phenylene))bis(oxy)bis(3-allylthio)-1-oxopropan-2-aminlum chloride

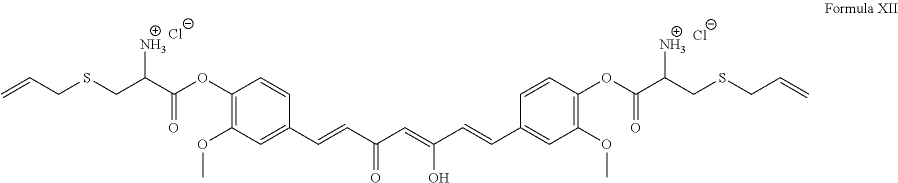 [SMILES: [Cl-] ; [Cl-] ; C=CCSCC([NH3+])C(=O)Oc1ccc(/C=C/C(=O)/C=C(O)/C=C/c2ccc(OC(=O)C([NH3+])CSCC=C)c(OC)c2)cc1OC]

The preparation involves the following steps:

(i) 3-(allylthio)-2-aminopropanoic acid (S-Allylcysteine)

Allyl bromide (2.29 ml; 26.42 mmol) was added to a solution of L-cysteine (2 g, 16.51 mmol) in 2M NH₄OH (28 ml) and the reaction mixture was stirred at room temperature for 20 hours. The solution was concentrated under reduced pressure and the white solid formed was washed with ethanol. The white soild has melting point 184.1-188.8° C.

(ii) S-allyl-N-(tert-butoxycarbonyl)cysteine

A solution of di-tert-butylcarbonate (Boc₂O; 2.98 g; 13.64 mmol) in 3 ml of THF was added to a solution of S-Allylcysteine (2 g; 12.40 mmol) in THF (35 ml) and 1M K₂CO₃ (16 ml) and the reaction mixture was stirred under nitrogen for 20 hours. At the end of the reaction, the salts were filtered, THF was evaporated and the residue was taken up with water and extracted twice with ethyl ether. The aqueous phase was acidified with acetic acid to pH 4 and extracted with ethyl acetate. The organic phase was washed with brine and dried with anhydrous sodium sulphate and the solvent was evaporated, yielding a yellow oil, which crystallized in freezer overnight. A white solid with melting point 48.7-51.4° C. was obtained.

(iii) ((1E,3Z,6E)-3-hydroxy-5-oxohepta-1,3,6-triene-1,7-diyl))bis(2-methoxy-4,1-phenylene)bis(3-(allylthio)-2-(tert-butoxycarbonyl)amino)propanoate

Compound prepared in step (ii) (795 mg; 3.040 mmol) was dissolved in 4 ml of CHCl₃. Then, EDC-HCl (637 mg; 3.325 mmol), DMAP (11.6 mg; 0.095 mmol) and a solution of curcumin (350 mg; 0.950 mmol) in CHCl₃ (4 ml) were added. The reaction mixture was stirred at room temperature, under nitrogen for 22 hours. At the end of the reaction, the organic phase was washed 3 times water and 3 times with brine. The organic phase dried with anhydrous sodium sulphate and finally was evaporated. The residue was purified on silica gel column, using cyclohexane/AcOEt (82:18) as eluent.

An orange solid was obtained.

(iv) 1,1′-((((1E,3Z,6E)-3-hydroxy-5-oxohepta-1,3,6-triene-1,7-diyl)bis(2-methoxy-4,1-phenylene))bis(oxy)bis(3-allylthio)-1-oxopropan-2-aminium chloride

Compound (iii) (40 mg; 0.047 mmol) was dissolved in a solution of 3M HCl in dioxane (500 □l) and 28□l of water (as scavenger) at 0° C. Then, the reaction mixture was allowed to warm to room temperature and was stirred under nitrogen for 1 hour. At the end of the reaction, the acid and the solvent were evaporated under reduced pressure and the residue was washed several tomes with ethyl ether. An orange solid with melting point 120.8-124.3° C. was obtained.

¹H-NMR (300 MHz, DMSO-d₆): δ=8.80 (s, 4H, 2 —NH₂, collapsed with D₂O), 8.25 (s, 1H, —OH, collapsed with D₂O), 7.82 (d, 2H, J=15.95 Hz, =CH), 7.64 (dd, 2H, J=6.0 e 15.9 Hz, ArH), 7.42-7.17 (m, 4H, ArH), 7.00 (d, 2H, J=15.6 Hz, =CH), 6.20 (s, 1H, =CH), 5.88-5.69 (m, 2H, 2 —CH═CH₂), 5.18 (dd, 4H, J=29.1 e 16.7 Hz, 2 —CH═CH), 4.59 (t, 2H, J=5.55 Hz, 2 —CH), 3.82 (s, 6H, 2 —OCH₃), 3.30 (d, 4H, J=6.9 Hz, 2 —CH₂), 3.08-2.98 (m, 4H, 2 —CH₂) ppm.

### Example 11: Biological Activity

a) Citotoxicity

The citotoxicity of compounds described in examples 1 and 2 was tested with the test Neutral Red Uptake to determine the IC₅₀ values. To better define the cytotoxic potential also the lactate dehydrogenase (LDH) release was tested to verify the action on cell membrane permeability. Test were performed according to DB-ALM Protocol n L39Balb3T3 Red Uptake cytotoxicity and ISO 10993-5:2009.

The results for compound of example 1 showed that the IC₅₀ is equal to 0.113 mM, while the results obtained by the LDH assay caused release of 13.2-13.0% of total enzyme contained in the cells after 14 hours exposition respectively at 0.5-0.2 mM. The results have not highlighted a specific activity on cell membrane.

The results for compound of example 2 showed that the IC₅₀ is >3 mM while the results obtained by the LDH assay caused release of 12.3% of total enzyme contained in the cells after 14 hours' exposition at 1 mM. The results have shown some slight activity on cell membrane at 1M concentration. Overall the substance is very biocompatible.

b) Scavenger Activity and Antioxidant Activity

The antioxidant power of compounds described in examples 1 and 2 was tested in two different tests, one for the scavenger activity and one for the antioxidant activity. The method is based on the fluorimetric probe 2′,7′-dichlorofluorescein diacetate (DCA) that becomes fluorescent only when it is oxidized by free radicals.

The results obtained prove that the compound of example 1 at 0.004 mM has an excellent scavenger activity, able to reduce the formation of the free radicals on average of 41% after 5 treatments with UVA.

The results also proved a good antioxidant efficacy with a 0.1 mM effective dose for inhibiting more than 50% free radical formation after 5 treatments UVA. The results showed that the compound can be a good help to fight the oxidative stress caused by internal (aerobic metabolism) and external factors (for ex. Environmental pollution)

For the compound of example 2, the results showed a mild scavenger action with 23.5% reduction of free radical formation after first UVA treatment.

The antioxidant activity of the compound of example 2, as expected, is weaker than for compound of example 1 with inhibition of free radical formation of 25.6% at 0.025 mM.

### Example 12. In Vitro Permeation of Compound of Example 1

The in vitro skin permeation experiments were developed and validated according to the Organization for Economic Cooperation and Development adopted guideline 428 (OECD guideline for the testing of chemicals. Skin absorption: in vitro method. Guideline 428 (Paris, April 2004, updated January 2011)).

Briefly, static Franz diffusion cells were set up with human skin samples according with OECD and SCSS guidelines. A simple formulation containing 1% of compound of example 1 in solution was applied topically on human skin samples (5 mg/cm2) for 24 hours in an unocclusive conditions. At the end of the exposure time the Franz diffusion cells were dismantled and the distribution of the compound was measured on the skin surface, in the stratum corneum and in the rector fluid. An extraction method was developed and the day after the extraction process, all the samples were filtered through 0.22 μm membrane filter into HPLC vials. The samples of compounds of example 1, together with known concentration standards were assayed by HPLC-UV (Waters, USA). The average amount measured in the dermis was 2% of applied dose and in the receptor fluid was 3% of the applied dose. Therefore, the calculated average dermal absorption for compound 1 was 5%.

### Example 13. Formulation: Fluid and Highly Hydrating Cream

### Composition

| | |
|---|---|
| Compound of Example 2 | 1% |
| PEG-9 Dimethicone | 5% |
| Dimethicone, Dimethicone/vinyl Dimethicone crosspolymer | 4% |
| Dimethicone | 24% |
| Butylene glycol | 3% |
| Phenoxyethanol | 0.5% |
| PEG-26-PPG-30 Phosphate | 0.5% |
| Ammonium Acryloyldimetylmethyltaurate/VP Copolymer | 0.5% |
| Silica | 1% |
| Lauroyl lysine | 0.5% |
| Water | 60% |

### Manufacturing Process:

Weight the compound of example 2, PEG-9 dimethicone, Dimethicone, Dimethicone/vinyl Dimethicone crosspolymer, Phenoxyethanol, Parfum. Add these components to water and Butylene glycol and disperse in the mixture Ammonium Acryloyldimetylmethyltaurate/VP Copolymer together with silica and lauryllysine using a homogeneizer.

### Example 14. Formulation: Serum for Sensitive Skin

### Composition

| | |
|---|---|
| Compound of example 1 | 3% |
| Dimethicone, Dimethicone/PEG-10/15 | 3% |
| Methyl trimethicone, DImethycone/vinyl dimethicone | 4% |
| crosspolymer | |
| Lauryl PEG-9 Polydimethylsiloxyethyl Dimethicone | 1% |
| Dimethicone | 8% |
| Sodium chloride | 0.5% |
| Butylene glycol | 5% |
| Glyceryl caprylate, ethylhexylglycerin | 0.5% |
| Water | 75% |

### Manufacturing Process

Add fluid silicones to the crosspolymers and compound of example 1 until system is homogenous. Prepare the aqueous phase containing sodium chloride, and add slowly to the silicones mixture. When water addition is completed add butyle glycol and glyceryl capylate, ethylhexylglycerin under vigorous stirring.

### Example 15. Formulation: Fluid BB Cream

This is a BB oil in water (O/W) fluid cream

### Composition

| | |
|---|---|
| Compound of example 2 | 0.5% |
| Glyceryl stearate, PEG-100 stearate | 4% |
| Cetearyl Alcohol | 0.5% |
| Tridecylstearate, Neopentyl glycol, Dicaprylate/dicaprate, | 14% |
| Trydecyl trimellitate (Lipovol MOS-70 ®) | |
| Ethylhexyl methoxycinnamate | 7% |
| Butyl methoxydibenzoylmethane | 3% |
| Water | 60.3% |
| Xantan gum | 0.2% |
| Butylene glycol | 2% |
| Sodium stearoyl glutamate | 0.5% |
| CI 77891, Talc, Magnesium stearate, Aluminium hydroxide, | 6% |
| Mica Stearic acid, (WPC-2039 Nikko ®) | |
| Silica | 1% |
| Phenoxyethanol | 1% |

### Manufacturing Process

Weight the compound of example 2, Glyceryl stearate, PEG-100 stearate, Cetearyl Alcohol, Lipovol MOS-70®), Ethylhexyl methoxycinnamate, Butyl methoxydibenzoylmethane and heat up to 70° C. (Phase 1). Disperse xanthan gum into water and butylene glycol, and then dissolve Sodium stearoyl glutamate heating up to 70° C. Add the pigment and silica, mix well until the mixture is homogeneous (Phase 2).

Add Phase 1 into Phase 2 and homogenize. After cooling at 40° C., add phenoxyethanol. Bring to final volume with water and adjust to pH 6.

### Example 16. Formulation: Hand Cream

| | |
|---|---|
| Compound of example 2 | 2% |
| Cetearyl alcohol, Polyglyceryl-10 stearate, Polyglyceryl-6 | 3% |
| tristearate, Hydroxypropyl guar (Emulprogress 57 ®) | |
| Cetearyl alcohol | 2% |
| Ethylhexyl palmitate | 6% |
| Caprylic/capric triglyceride | 5% |
| Ascorbyl palmitate | 0.1% |
| Butylene glycol | 4% |
| Glyceryl caprylate, ethylhexylglycerin | 0.4% |
| Water | 77.5% |

### Manufacturing Process

Weight the compound of example 2, Emulprogress 57®, Cetearyl alcohol, Ethylehylpalmitate, caprylic/capric triglyceride, and ascorbylpalmitate and heat up to 70° C. Add water and mix well with a homogenizer. Cool down to 40° C. and add a water solution of butylene glycol and glyceryl caprylate, ethylexhylglycerin. Adjust, if necessary the pH of the preparation to pH 6.0-6.5.

### Example 17. Formulation: Suncare Primer

### Composition

Phase 1

| | |
|---|---|
| Compound of example 1 | 0.5% |
| Caprylyl Methicone, PEG 12 Dimethicone/PPG-20 | 35% |
| crosspolymer | |
| Caprylylmethicone | 5% |

Phase 2

| | |
|---|---|
| Butylmethoxydibenzoylmethane | 1% |
| Ethylhexyl salicylate | 5% |
| Octocrylene | 2% |
| Caprylic/capric triglyceride | 4% |
| Dicaprylyl carbonate | 3% |

Phase 3

| | |
|---|---|
| Glycerin | 31% |
| Water | 10.5% |
| Matrixylil 3000 ® | 3% |

### Manufacturing Process

Mix phase 1 components until homogeneous. In a separate vessel add the components of phase 2 and slowly add phase 2 to phase 1 under vigorous mixing.

Mix phase 3 and slowly add this phase to the previous mixed phases 1 and 2 with turbulent mixing.

Although certain preferred embodiments have been defined on the present application, their purpose is illustrative only and is not intended to limit the scope of the invention. The claims accompanying this description are considered to cover all such embodiments and equivalents thereof.

Finally, modifications of the present invention which are obvious to one skilled in the art, such as adding or removing non-essential elements to its embodiment, can be made without departing from the scope and spirit of the invention.

1-29. (canceled)

30. Ester compounds characterized to have the formula I as follows: 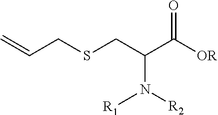
or a pharmaceutically acceptable salt or derivatives thereof, wherein:
R can be an unsubstituted, branched or unbranched, unsaturated or saturated C1-C6 alkyl, allyl, or aryl group ester, and R1=R2=H;
wherein R can be an antioxidant compound such as: flavonoid or a derivative thereof such as resveratrol (mono- di-, tri-substituted on the oxydryl functions), naringenin, catechins, quercetin, derivatives of gallic acid (such as catechins dodecyl gallate and propyl gallate), or tocopherol or derivatives thereof including the synthetic analogues butylhydroxyanisole (BHA) and butylhydroxytolune (BHT), or carotenoids compounds such as curcumin, or vitamin C or derivatives thereof such as ascorbyl palmitate, or triterpenoid such as celastrol.

31. Compound according to claim 30 wherein the compound is selected from the group comprising: E)-1,1′-((5-(4-((3-(allylthio)-2-ammoniopropanoyl)oxy)styryl)-1,3-phenylene)bis(oxy))bis(3-(allylthio)-1-oxopropan-2-aminium) chloride; 3-(allylthio)-1-methoxy-1-oxopropan-2-aminium chloride; 1,1′-((((1E,3Z,6E)-3-hydroxy-5-oxohepta-1,3,6-triene-1,7-diyl)bis(2-methoxy-4,1-phenylene))bis(oxy)bis(3-allylthio)-1-oxopropan-2-aminium chloride.

32. Compound according to claim 30 having the formula III as follows:

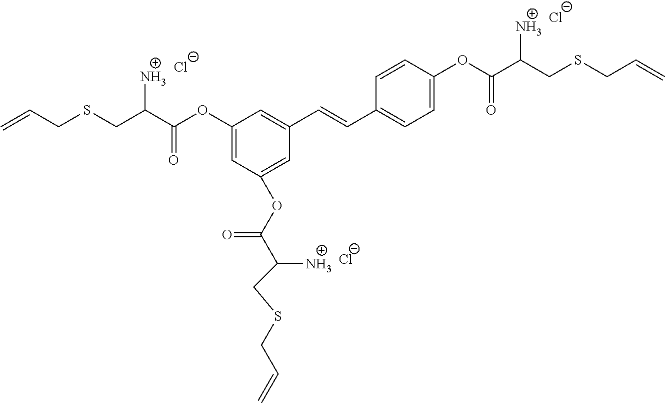 [SMILES: [Cl-] ; C=CCSCC([NH3+])C(=O)Oc1ccc(/C=C/c2cc(OC(=O)C([NH3+])CSCC=C)cc(OC(=O)C([NH3+])CSCC=C)c2)cc1 ; [Cl-] ; [Cl-]]

33. Compound according to claim 30 having the formula XII as follows:

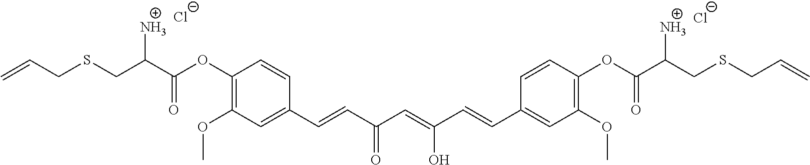 [SMILES: [Cl-] ; [Cl-] ; C=CCSCC([NH3+])C(=O)Oc1ccc(/C=C/C(=O)/C=C(O)/C=C/c2ccc(OC(=O)C([NH3+])CSCC=C)c(OC)c2)cc1OC]

34. Amide compounds characterized to have the formula II: 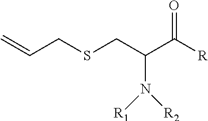
or a pharmaceutically acceptable salt or derivatives thereof, wherein: R is —OH group or —O— unsubstituted, branched or unbranched, unsaturated or saturated C1-C6 alkyl, allyl, or aryl group or pharmaceutically acceptable salt thereof, and R1≠R2 wherein R1 or R2 is H or an C1-C6 acyl group, wherein said C1-C6 acyl group is linked to an unsubstituted, branched or unbranched, unsaturated or saturated C1-C6 alkyl, allyl, or aryl group, and when R1 different from H then R2 is H;
wherein R1 or R2 is H or derivatives of pantothenic acid, folic acid, hyaluronic acid, pyroglutammic acid, gluconic acid, omega 3 fatty acids (such as docosahexaenoic acid), vitamin C or derivatives thereof such as ascorbyl palmitate, hydroxyacids (such as glycolic acid, lactic acid, citric acid, mandelic acid).

35. Compound according to claim 34 wherein the compound is selected from the group comprising: L-Pyroglutamyl-(R)-3-Allylsulfanyl-2-amino-propionic acid methyl ester; methyl (E)-S-allyl-N-(3-(3,4-dihydroxyphenyl)acryloyl) cysteinate; (E)-S-allyl-N-(3-(4-hydroxy-3-methoxyphenyl) acryloyl)cysteinate; methyl S-allyl-N-(2-hydroxy-2-phenylacetyl)cysteinate; S-allyl-N-((2R,4aS,6aS,12bR,14aS)-10-hydroxy-2,4a,6a,9,12b,14a-hexamethyl-11-oxo-1,2,3,4,4a,5,6,6a,11,12b,13,14,14a,14b-tetradecahyd ropicene-2-carbonyl)cysteinate; methyl S-allyl-N-(2-hydroxyacetyl)cysteinate; N5-(3-(allylthio)-1-methoxy-1-oxopropan-2-yl)-N2-(4-(((2-amino-4-hydroxypteridin-6-yl)methyl)amino)benzoyl)glutamine.

36. Compound according to claim 34 comprising the formula IV as follows:

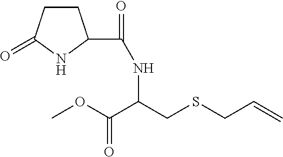 [SMILES: C=CCSCC(NC(=O)C1CCC(=O)N1)C(=O)OC]

37. Compound according to claim 34 comprising the formula VI as follows:

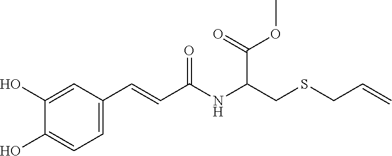 [SMILES: C=CCSCC(NC(=O)/C=C/c1ccc(O)c(O)c1)C(=O)OC]

38. Compound according to claim 34 comprising the formula VII as follows:

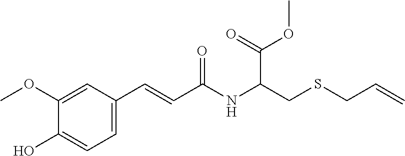 [SMILES: C=CCSCC(NC(=O)/C=C/c1ccc(O)c(OC)c1)C(=O)OC]

39. Compound according to claim 34 comprising the formula VIII as follows:

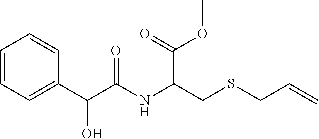 [SMILES: C=CCSCC(NC(=O)C(O)c1ccccc1)C(=O)OC]

40. Compound according to claim 34 comprising the formula IX as follows:

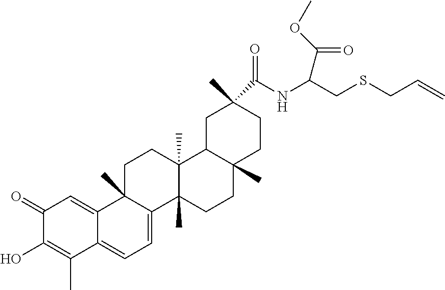 [SMILES: C=CCSCC(NC(=O)[C@]1(C)CC[C@]2(C)CC[C@]3(C)C4=CC=C5C(=CC(=O)C(O)=C5C)[C@]4(C)CC[C@@]3(C)C2C1)C(=O)OC]

41. Compound according to claim 34 comprising the formula X as follows:

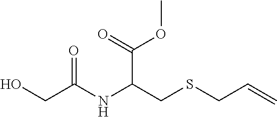

42. Compound according to claim 34 comprising the formula XI as follows:

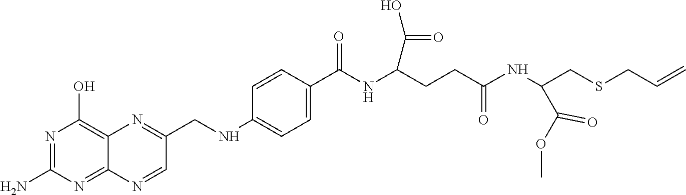 [SMILES: C=CCSCC(NC(=O)CCC(NC(=O)c1ccc(NCc2cnc3nc(N)nc(O)c3n2)cc1)C(=O)O)C(=O)OC]

43. Process for production of a compound according to claim 30 wherein it comprises the steps of:
(a) solubilize the (S)-allylcysteine derivative in at least one solvent selected from the group consisting of tetrahydrofurane (THF), CH₂Cl₂, dichloromethane, hydrogen chloride, dioxane, dimethyl formamide (DMF), water, CH₃CN, dimethyl sulfoxide (DMSO) and CHCl₃;
(b) add the alcoholic or carboxylic compound for the preparation of the ester or amide;
(c) add at least one coupling agent selected from the group consisting of N,N′-dicyclohexylcarbodiimide (DCC), triethylamine (TEA), 1-hydroxy benzotriazole (HOBt), N-hydroxysuccinimide (NHS), 4-N,N-dimethylaminopyridine (DMAP), 1-Ethyl-3-(3-dimethylamino propyl) carbodiimide hydrochloride (EDC-HCl) and diisopropyilethylamine (DIPEA); and
(d) purify the resulting ester or amide by chromatography or crystallization.

44. Composition comprising:
(i) 0.001-50% w/w of at least one allyl-S-cystein derivative as defined on any one of claims 1 to 15 and optionally one or more additional active compounds;
(ii) 50-99.999% w/w of at least one cosmetically acceptable excipient selected from silicones, emulsifiers, surfactants, thickeners, powders, film formers, rheology modifying agents, propellants, fragrance, opacifiers, preservatives, colorants, pigments, buffers, chelating agents, sensory enhancers and combinations thereof.

45. Composition according to claim 44 wherein it is in the form of an aerosol, gel, cream, milk, lotion, serum, oil, scrub, powder, mask, primer, toner, soap, cleanser, shower gel, make up, concealer, pressed powder, mascara or a lipstick.

46. Composition according to claim 44 wherein it is in the form of a cream, serum or primer.

47. Composition according to claim 44 wherein it is incorporated into a wrap or film, a mask, a patch, a cloth or blanket, a pad, a sheet, a wipe, a pen or the like.

48. Composition according to claim 44 comprising the fluid and hydrating cream of Example 13.

49. Composition according to claim 44 comprising the serum of Example 14.

50. Composition according to claim 44 comprising the fluid BB cream of Example 15.

51. Composition according to claim 44 comprising the hand cream of Example 16.

52. Composition according to claim 44 comprising suncare primer of Example 17.

53. Method for treatment and/or amelioration of the effects of skin aging comprising administration of at least one compound according to claim 30 to a subject.

54. Method according to claim 53 wherein the administration is made by a cosmetic or dermatological composition.

55. Use of at least one compound according to claim 30 wherein it is to prepare a cosmetic or dermatological composition.

56. Use of at least one compound according to claim 30 wherein it is to prepare a composition for the treatment and/or amelioration of the effects of skin aging.
